SEQUENCE LISTING (19 sequences)

SEQ ID NO: 1 - protein, 261 aa, Homo sapiens
  SOURCE 1..261: /mol_type=“protein” /organism=“Homo sapiens”
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 16 aa, Homo sapiens
  SOURCE 1..16: /mol_type=“protein” /organism=“Homo sapiens”
TKFMLCAGRWTGGKST

SEQ ID NO: 3 - protein, 31 aa, Homo sapiens
  SOURCE 1..31: /mol_type=“protein” /organism=“Homo sapiens”
GDSGGPLVCNGVLQGITSWGSEPCALPERPS

SEQ ID NO: 4 - protein, 12 aa, Homo sapiens
  SOURCE 1..12: /mol_type=“protein” /organism=“Homo sapiens”
PQKVTKFMLCAG

SEQ ID NO: 5 - protein, 124 aa, Homo sapiens
  SOURCE 1..124: /mol_type=“protein” /organism=“Homo sapiens”
IRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQ

SEQ ID NO: 6 - protein, 23 aa, Homo sapiens
  SOURCE 1..23: /mol_type=“protein” /organism=“Homo sapiens”
RHSLFHPEDTGQVFQVSHSFPHP

SEQ ID NO: 7 - protein, 25 aa, Homo sapiens
  SOURCE 1..25: /mol_type=“protein” /organism=“Homo sapiens”
YDMSLLKNRFLRPGDDSSHDLMLLR

SEQ ID NO: 8 - protein, 14 aa, Homo sapiens
  SOURCE 1..14: /mol_type=“protein” /organism=“Homo sapiens”
MLLRLSEPAELTDA

SEQ ID NO: 9 - protein, 21 aa, Homo sapiens
  SOURCE 1..21: /mol_type=“protein” /organism=“Homo sapiens”
VVFLTLSVTWIGAAPLILSRI

SEQ ID NO: 10 - protein, 94 aa, Homo sapiens
  SOURCE 1..94: /mol_type=“protein” /organism=“Homo sapiens”
KNRFLRPGDDSSHDLMLLRLSEPAELTDAVKVMDLPTQEPALGTTCYASGWGSIEPEEFLTPKKLQCVDLHVISNDVCAQVHPQKVTKFMLCAG

SEQ ID NO: 11 - protein, 237 aa, Homo sapiens
  SOURCE 1..237: /mol_type=“protein” /organism=“Homo sapiens”
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 12 - protein, 1205 aa, Bos taurus
  SOURCE 1..1205: /mol_type=“protein” /organism=“Bos taurus”
MGNLKSVGQEPGPPCGLGLGLGLGLCGKQGPASPAPEPSRAPAPATPHAPDHSPAPNSPTLTRPPEGPKFPRVKNWELGSITYDTLCAQSQQDGPCTPRCCLGSLVLPRKLQTRPSPGPPPAEQLLSQARDFINQYYSSIKRSGSQAHEERLQEVEAEVASTGTYHLRESELVFGAKQAWRNAPRCVGRIQWGKLQVFDARDCSSAQEMFTYICNHIKYATNRGNLRSAITVFPQRAPGRGDFRIWNSQLVRYAGYRQQDGSVRGDPANVEITELCIQHGWTPGNGRFDVLPLLLQAPDEAPELFVLPPELVLEVPLEHPTLEWFAALGLRWYALPAVSNMLLEIGGLEFSAAPFSGWYMSTEIGTRNLCDPHRYNILEDVAVCMDLDTRTTSSLWKDKAAVEINLAVLHSFQLAKVTIVDHHAATVSFMKHLDNEQKARGGCPADWAWIVPPISGSLTPVFHQEMVNYILSPAFRYQPDPWKGSATKGAGITRKKTFKEVANAVKISASLMGTLMAKRVKATILYASETGRAQSYAQQLGRLFRKAFDPRVLCMDEYDVVSLEHEALVLVVTSTFGNGDPPENGESFAAALMEMSGPYNSSPRPEQHKSYKIRFNSVSCSDPLVSSWRRKRKESSNTDSAGALGTLRFCVFGLGSRAYPHFCAFARAVDTRLEELGGERLLQLGQGDELCGQEEAFRGWAKAAFQASCETFCVGEEAKAAAQDIFSPKRSWKRQRYRLSTQAEGLQLLPGLIHVHRRKMFQATVLSVENLQSSKSTRATILVRLDTAGQEGLQYQPGDHIGICPPNRPGLVEALLSRVEDPPPPTESVAVEQLEKGSPGGPPPSWVRDPRLPPCTLRQALTFFLDITSPPSPRLLRLLSTLAEEPSEQQELETLSQDPRRYEEWKWFRCPTLLEVLEQFPSVALPAPLLLTQLPLLQPRYYSVSSAPNAHPGEVHLTVAVLAYRTQDGLGPLHYGVCSTWLSQLKTGDPVPCFIRGAPSFRLPPDPYVPCILVGPGTGIAPFRGFWQERLHDIESKGLQPAPMTLVFGCRCSQLDHLYRDEVQDAQERGVFGRVLTAFSREPDSPKTYVQDILRTELAAEVHRVLCLERGHMFVCGDVTMATSVLQTVQRILATEGDMELDEAGDVIGVLRDQQRYHEDIFGLTLRTQEVTSRIRTQSFSLQERHLRGAVPWAFDPPGPDTPGP

SEQ ID NO: 13 - protein, 1203 aa, Homo sapiens
  SOURCE 1..1203: /mol_type=“protein” /organism=“Homo sapiens”
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 14 - protein, 4 aa, Bos taurus
  SOURCE 1..4: /mol_type=“protein” /organism=“Bos taurus”
PWAF

SEQ ID NO: 15 - protein, 4 aa, Bos taurus
  SOURCE 1..4: /mol_type=“protein” /organism=“Bos taurus”
GAVP

SEQ ID NO: 16 - protein, 21 aa, Bos taurus
  SOURCE 1..21: /mol_type=“protein” /organism=“Bos taurus”
RHLRGAVPWAFDPPGPDTPGP

SEQ ID NO: 17 - protein, 12 aa, Bos taurus
  SOURCE 1..12: /mol_type=“protein” /organism=“Bos taurus”
AFDPPGPDTPGP

SEQ ID NO: 18 - protein, 11 aa, Bos taurus
  SOURCE 1..11: /mol_type=“protein” /organism=“Bos taurus”
HLRGAVPWAFD

SEQ ID NO: 19 - protein, 20 aa, Bos taurus
  SOURCE 1..20: /mol_type=“protein” /organism=“Bos taurus”
HLRGAVPWAFDPPGPDTPGP

(19) United States
(12) Patent Application Publication
(10) Pub. No.: US 2013/0064860 A1
(43) Pub. Date: Mar. 14, 2013

(54) Combination pharmaceutical composition and methods of theating genitourinary system disorders

(71) Applicant: Oleg Iliich Epshtein, Moscow (RU)

(72) Inventor: Oleg Iliich Epshtein, Moscow (RU)

(21) Appl. No.: 13/135,898

(22) Filed: Jul. 15, 2011

(30) Foreign Application Priority Data
Jul. 15, 2010 (RU) 2010129294
Jul. 15, 2010 (RU) 2010129295
Jul. 1, 2011 (RU) 2011127053

(51) Int. Cl. A61K 39/395; A61P 15/10; A61P 15/00; A61K 9/00

(52) U.S. Cl. 424/400; 424/158.1; 424/152.1; 424/146.1; 424/139.1

(57) ABSTRACT

The invention provides a pharmaceutical composition comprising a) an activated-potentiated form of an antibody to prostate-specific antigen, and b) an activated-potentiated form of an antibody to endothelial NO-synthase. Various embodiments and variants are provided.

The invention provides methods of treating benign prostatic hyperplasia and erectile dysfunctions and various methods of administration comprising administering a pharmaceutical composition comprising a) an activated-potentiated form of an antibody to prostate-specific antigen, and b) an activated-potentiated form of an antibody to endothelial NO-synthase.

### FIELD

The preset invention relates to a combination pharmaceutical compositions and method of treating genitourinary system disorders.

### BACKGROUND

The invention refers to the field of medicine and can be used to treat genitourinary system disorders, including prostate gland disorders, including benign prostatic hyperplasia of I and II degree, acute and chronic prostatitis, and erectile dysfunction of various origins.

Nitric oxide (NO) is a gaseous molecule that has been shown to acts in the signaling of different biological processes. Endothelium-derived NO is a key molecule in regulation of vascular tone and its association with vascular disease has long been recognized. NO inhibits many processes known to be involved in the formation of atherosclerotic plaque, including monocyte adhesion, platelet aggregation and vascular smooth muscle cell proliferation. Another important role of endothelial NO is the protection of the vascular wall from the oxidative stress induced by its own metabolic products and by the oxidation products of lipids and lipoproteins. Endothelial dysfunction occurs at very early stages of atherosclerosis. It is therefore possible that deficiency in local NO availability could be a final common pathway that accelerates atherogenesis in humans. In addition to its role in the vascular endothelium, NO availability has been shown to modulate metabolism of lipoproteins.

Negative correlation has been reported between plasma concentrations of NO metabolic products and plasma total and Low Density Lipoprotein [LDL] cholesterol levels while High Density Lipoprotein [HDL] improves vascular function in hypercholesterolaemic subjects. The loss of NO has considerable effect on the development of the disease. Diabetes mellitus is associated with increased rates of morbidity and mortality caused primarily by the accelerated development of atherosclerotic disease. Moreover, reports show that diabetics have impaired lung functions. It has been proposed that insulin resistance leads to airway inflammation. Habib et al., Nitric Oxide Measurement From Blood To Lungs, Is There A Link? Pak J Physiol 2007; 3(1).

Nitric oxide is synthesized by the endothelium from L-arginine by nitric oxide synthase (NO synthase). NO synthase occurs in different isoforms, including a constitutive form (cNOS) and an inducible form (iNOS). The constitutive form is present in normal endothelial cells, neurons and some other tissues.

Prostate-specific antigen (PSA), an antigen discovered in the 1970s and introduced to urological practice about 15 years ago. Although it is widely used as the most sensitive marker available so far for screening, diagnosis and monitoring human prostate cancer progression as well as response to therapy, discoveries over the past decade have unequivocally indicated that the original antigen PSA is no longer prostate-specific, shedding light on the multifunctional behaviour of this ‘novel’ serine protease. The glandular kallikrein gene family is composed of three genes, localized on chromosome 19q13.3-q13.4; the KLK-3 gene locus encodes the extracellular serine protease PSA, which has also been named human glandular kallikrein 3 (hK3). In the prostate, PSA expression is localized to the differentiated, secretory columnar cells of the glandular epithelium. Biochemically, it is a 33 kDa single-chain glycoprotein with chymotrypsin-like activity that requires post-translational processing for its full proteolytic activity.

Although PSA is produced by the prostatic epithelial cells in relatively enormous amounts and its regulation is under the control of androgens and progestins, we do not have a good understanding of why this molecule is so abundantly expressed and what role it plays in prostatic physiology.

The currently most widely accepted physiological function of PSA relates to its ability to digest the seminogelins and fibronectin present in high concentrations in seminal plasma (produced by the seminal vesicles), thus liquefying the seminal clot shortly after ejaculation. The physiologic consequences of the cleavage of seminogelins are not known, although this process does increase sperm cell motility. Other investigators have reported that PSA can release a kinin-like substance that stimulates smooth muscle contraction by digesting a glycoprotein present in seminal vesicle fluid. Some researchers portray PSA as a cell growth inhibitor, an anticarcinogenic/antiangiogenic molecule, or as an inducer of apoptosis. PSA should be considered as a “cancer fighter” at the tissue level and as a “valuable messenger” (indicator) at the level of the systemic circulation, which can be used to either detect or monitor cancer. A few other reports have suggested that PSA is an insulin-like growth factor binding protein-3 (IGFBP-3) protease that, through its proteolytic action, releases free, bioactive insulin-like growth factor I (IGF-I) previously bound to IGFBP-3. IGF-I is a known mitogen of many cell types and a risk factor for prostate and breast carcinoma development. It has suggested that PSA may activate latent transforming growth factor-β or may cleave parathyroid hormone-related peptide. (Diamandis E P. Prostate-specific antigen: a cancer fighter and a valuable messenger? Clin Chem. 2000 July; 46(7):896-900.)

Treatment of prostate gland disorders based on ultra-low doses of antibodies to prostate specific antigen is known in the art (U.S. Pat. No. 7,582,294). However, this medication does not guarantee sufficient therapeutic effectiveness in treating genitourinary system disorders, accompanied by erectile problems of various origins (erectile dysfunctions).

The therapeutic effect of an extremely diluted form (or ultra-low form) of antibodies potentized by homeopathic technology (activated potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of treating diseases of viral etiology. See U.S. Pat. No. 7,572,441, which is incorporated herein by reference in its entirety.

The present invention is directed to a combination pharmaceutical composition and methods of its use in treatment of genitourinary system disorders, including benign prostatic hyperplasia of I and II degree, acute and chronic prostatitis, and erectile dysfunction of various origins.

The solution to the existing problem is presented in form of a combination pharmaceutical composition for treatment and prophylaxis of genitourinary system disorders which comprises activated-potentiated form of antibodies to prostate specific antigen (PSA) and activated-potentiated form of antibodies to endothelial NO synthase.

### SUMMARY

In one aspect, the invention provides a combination pharmaceutical composition comprising a) an activated-potentiated form of an antibody to prostate specific antigen and b) an activated-potentiated form of an antibody to endothelial NO synthase. In an embodiment, the combination pharmaceutical composition further comprises a solid carrier, wherein said activated-potentiated form an antibody to prostate specific antigen and said activated-potentiated form of an antibody to endothelial NO synthase are impregnated onto said solid carrier. In a variant, the combination pharmaceutical composition is in the form of a tablet.

Preferably, the combination pharmaceutical composition includes said activated-potentiated form of an antibody to prostate specific antigen which is in the form of a mixture of C12, C30, and C200 homeopathic dilutions. It is specifically contemplated that said mixture of C12, C30, and C200 homeopathic dilutions is impregnated onto a solid carrier.

Preferably, the combination pharmaceutical composition includes said activated-potentiated form of an antibody to endothelial NO synthase which is in the form of a mixture of C12, C30, and C200 homeopathic dilutions. It is specifically contemplated that said mixture of C12, C30, and C200 homeopathic dilutions is impregnated onto a solid carrier.

The activated-potentiated form of an antibody to prostate specific antigen may be a monoclonal, polyclonal or natural antibody. It is specifically contemplated that the activated-potentiated form of an antibody to prostate specific antigen is a polyclonal antibody. The activated-potentiated form of an antibody to endothelial NO synthase may be a monoclonal, polyclonal or natural antibody. It is specifically contemplated that the activated-potentiated form of an antibody to endothelial NO synthase is a polyclonal antibody. The invention provides activated-potentiated forms of antibodies to antigen(s) having sequences described in the specification and claimed in the appended claims.

In a variant, the combination pharmaceutical composition includes activated-potentiated form of an antibody to prostate specific antigen prepared by successive centesimal dilutions coupled with shaking of every dilution. In a variant, the combination pharmaceutical composition includes activated-potentiated form of an antibody to endothelial NO synthase prepared by successive centesimal dilutions coupled with shaking of every dilution. Vertical shaking is specifically contemplated.

In another aspect, the invention provides a method of treating genitourinary system disorders, said method comprising administering to a patient in need thereof a) an activated-potentiated form of an antibody to prostate specific antigen and b) an activated-potentiated form of an antibody to endothelial NO synthase in the form of combined pharmaceutical composition.

In an embodiment, the combination pharmaceutical composition is administered in the form of a solid oral dosage form which comprises a pharmaceutically acceptable carrier and said activated-potentiated form of an antibody to prostate specific antigen, impregnated onto said carrier, and said activated-potentiated form of an antibody to endothelial NO synthase, impregnated onto said carrier. In a variant, said solid oral dosage form is a tablet. Variants and embodiments are provided.

In accordance with the method aspect of the invention, the combination pharmaceutical composition may be administered in one to four unit dosage forms, each of the dosage form being administered from once daily to six times daily. In accordance with the method aspect of the invention, the combination pharmaceutical composition may be administered as follows:

1 pill 1 time/day;

1 pill 2 times/day;

1 pill 3 times/day;

1 pill 4 times/day;

1 pill 5 times/day;

1 pill 6 times/day;

2 pills 1 time/day;

2 pills 2 times/day;

2 pills 3 times/day;

2 pills 4 times/day;

2 pills 5 times/day;

2 pills 6 times/day;

3 pills 1 time/day;

3 pills 2 times/day;

3 pills 3 times/day;

3 pills 4 times/day;

4 pills 1 time/day;

4 pills 2 times/day;

4 pills 3 times/day.

All variants and embodiments described with respect to the composition aspect of the invention may be used with the method aspect of the invention.

Co-administration of the combination pharmaceutical composition with an additional active ingredient is specifically contemplated. In a variant, the additional active ingredient is approved for treatment of genitourinary system disorders. Variants and embodiments are contemplated.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³° and 100⁵° times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological ‘activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well-accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated-potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated’ form of the antibodies of the present invention is not attributable to the initial molecular form of the antibody. Preferred is the “activated-potentiated” form of antibody in liquid or solid form in which the concentration of the molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

In one aspect, the present invention provides a combination pharmaceutical composition comprising a) an activated-potentiated form of an antibody to prostate specific antigen and b) an activated-potentiated form of an antibody to endothelial NO synthase. As set forth herein above, each of the individual components of the combination is generally known for its own individual medical uses. However, the inventors of the present patent application surprisingly discovered that administration of the combination remarkably increases efficacy of the treatment of genitourinary system disorders. The claimed combination pharmaceutical composition of activated-potentiated antibodies to prostate specific antigen (PSA) and to endothelial NO synthase in a mixture ensures an unexpected synergetic therapeutic effect, confirmed by adequate experimental models and clinical studies, consisting of improved vascularization, increased antiadenoma (antiproliferative) effect and increased anti-inflammatory effect. The proposed medical product contributed to normalization of functional conditions of prostate and lower sections of urinary tract, improvement of uridinamic functions and a decrease of erectile dysfunctions, and contributes to normalization of PSA level. The proposed product can be used not only during a conservative therapy, but in patients with benign prostate hyperplasia, who underwent a surgical procedure to reduce the size of prostate gland, activate regenerative-reparative processes in patients, who underwent a surgical procedure to treat benign prostatic hyperplasia, reduces a possibility of post surgery complications.

In addition, the proposed technical solution improves quality of life in patients with benign prostatic hyperplasia (BPH), prostatitis and other prostate disorders, reduces dysuric disorder occurrences, producing an vegetative stabilizing effect and improves sperm producing properties. This technical result is justified by endothelial protective property of an activated-highly potent form of antibodies to endothelial NO synthase, that boosts antiproliferative and anitiflammatory activities of an activated potentiated form of antibodies to PSA, due to the effect of an activated-potentiated form of antibodies to endothelial NO synthase on transduction intracellular signals during their simultaneous and combined use as a comprehensive medication among others.

At the same time, the proposed invention is characterized by a wide range of therapeutic effectiveness and can be used to treat a variety of genitourinary system disorders, accompanied by prostate gland problems and erectile dysfunctions, as well as part of complex therapy.

The pharmaceutical composition of the invention expands the arsenal of preparations available for the treatment and prophylaxis of genitourinary system disorders.

In another aspect, the invention provides a method of treating a genitourinary system disorder, said method comprising administering to a patient in need thereof a) an activated-potentiated form of an antibody to prostate specific antigen and b) an activated-potentiated form of an antibody to endothelial NO synthase in the form of combined pharmaceutical composition.

In one variant, the genitourinary system disorder includes prostate gland disorders, including benign prostatic hyperplasia of I and II degree, acute and chronic prostatitis, and erectile dysfunction of various origins.

In one variant the genitourinary system disorders is prostate gland disorder.

In one variant of this aspect of the invention the prostate gland disorder is benign prostatic hyperplasia.

In another variant of this aspect of the invention the prostate gland disorder is benign prostatic hyperplasia of II degree.

In another variant of this aspect of the invention the prostate gland disorder is acute or chronic prostatitis.

In another variant the genitourinary system disorders is erectile dysfunction of various origins.

In accordance with the method aspect of the invention, the combination pharmaceutical composition may be administered in one to four unit dosage forms, each of the dosage form being administered from once daily to six times daily. In accordance with the method aspect of the invention, the combination pharmaceutical composition may be administered as follows:

1 pill 1 time/day;

1 pill 2 times/day;

1 pill 3 times/day;

1 pill 4 times/day;

1 pill 5 times/day;

1 pill 6 times/day;

2 pills 1 time/day;

2 pills 2 times/day;

2 pills 3 times/day;

2 pills 4 times/day;

2 pills 5 times/day;

2 pills 6 times/day;

3 pills 1 time/day;

3 pills 2 times/day;

3 pills 3 times/day;

3 pills 4 times/day;

4 pills 1 time/day;

4 pills 2 times/day;

4 pills 3 times/day.

The pharmaceutical composition of the present invention for the purpose of treatment of genitourinary system disorders contains active components in volume primarily in 1:1 ratio.

The medical product is prepared mainly as follows.

The combination pharmaceutical composition in accordance with the present invention may be in the liquid form or in solid form. Each of the activated potentiated forms of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R. —2005—Vol. 14. —N 1-2. P. 33-55, both incorporated herein by reference.

Monoclonal antibodies may be obtained, e.g., by means of hybridoma technology. The initial stage of the process includes immunization based on the principles already developed in course of polyclonal antisera preparation. Further stages of work involve production of hybrid cells generating clones of antibodies with identical specificity. Their separate isolation is performed using the same methods as in case of polyclonal antisera preparation.

Polyclonal antibodies may be obtained via active immunization of animals. For this purpose, for example, suitable animals (e.g. rabbits) receive a series of injections of the appropriate antigen: prostate specific antigen and endothelial NO synthase. The animals' immune system generates corresponding antibodies, which are collected from the animals in a known manner. This procedure enables preparation of a monospecific antibody-rich serum.

If desired, the serum containing antibodies may be purified, e.g., using affine chromatography, fractionation by salt precipitation, or ion-exchange chromatography.

The resulting purified, antibody-enriched serum may be used as a starting material for preparation of the activated-potentiated form of the antibodies. The preferred concentration of the resulting initial solution of antibody in the solvent, preferably, water or water-ethyl alcohol mixture, ranges from about 0.5 to about 5.0 mg/ml. The preferred procedure for preparing each component is the use of the mixture of three aqueous-alcohol dilutions of the primary matrix solution of antibodies diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the combination of the invention is polyclonal antibodies to prostate specific antigen and endothelial NO synthase an initial (matrix) solution with concentration of 0.5 to 5.0 mg/ml is used for the subsequent preparation of activated-potentiated forms.

To prepare the pharmaceutical composition preferably polyclonal antibodies to prostate specific antigen and endothelial NO synthase are used.

Polyclonal antibodies to endothelial NO synthase are obtained using adjuvant as immunogen (antigen) for immunization of rabbits and whole molecule of bovine endothelial NO synthase of the following sequence:

```
SEQ.ID. NO. 1
Met Gly Asn Leu Lys Ser Val Gly Gln Glu Pro Gly Pro Pro Cys
  1 5 10 15
Gly Leu Gly Leu Gly Leu Gly Leu Gly Leu Cys Gly Lys Gln Gly
  16 20 25 30
Pro Ala Ser Pro Ala Pro Glu Pro Ser Arg Ala Pro Ala Pro Ala
  31 35 40 45
Thr Pro His Ala Pro Asp His Ser Pro Ala Pro Asn Ser Pro Thr
  46 50 55 60
Leu Thr Arg Pro Pro Glu Gly Pro Lys Phe Pro Arg Val Lys Asn
  61 65 70 75
Trp Glu Leu GLys er Ile Thr Tyr Asp Thr Leu Cys Ala Gln Ser
  76 80 85 90
Gln Gln Asp Gly Pro Cys Thr Pro Arg Cys Cys Leu GLys er Leu
  91 95 100 105
Val Leu Pro Arg Lys Leu Gln Thr Arg Pro Ser Pro Gly Pro Pro
106 110 115 120
Pro Ala Glu Gln Leu Leu Ser Gln Ala Arg Asp Phe Ile Asn Gln
121 125 130 135
Tyr Tyr Ser Ser Ile Lys Arg Ser GLys er Gln Ala His Glu Glu
136 140 145 150
Arg Leu Gln Glu Val Glu Ala Glu Val Ala Ser Thr Gly Thr Tyr
151 155 160 165
His Leu Arg Glu Ser Glu Leu Val Phe Gly Ala Lys Gln Ala Trp
166 170 175 180
Arg Asn Ala Pro Arg Cys Val Gly Arg Ile Gln Trp Gly Lys Leu
181 185 190 195
Gln Val Phe Asp Ala Arg Asp Cys Ser Ser Ala Gln Glu Met Phe
196 200 205 210
Thr Tyr Ile Cys Asn His Ile Lys Tyr Ala Thr Asn Arg Gly Asn
211 215 220 225
Leu Arg Ser Ala Ile Thr Val Phe Pro Gln Arg Ala Pro Gly Arg
226 230 235 240
Gly Asp Phe Arg Ile Trp Asn Ser Gln Leu Val Arg Tyr Ala Gly
241 245 250 255
Tyr Arg Gln Gln Asp GLys er Val Arg Gly Asp Pro Ala Asn Val
256 260 265 270
Glu Ile Thr Glu Leu Cys Ile Gln His Gly Trp Thr Pro Gly Asn
271 275 280 285
Gly Arg Phe Asp Val Leu Pro Leu Leu Leu Gln Ala Pro Asp Glu
286 290 295 300
Ala Pro Glu Leu Phe Val Leu Pro Pro Glu Leu Val Leu Glu Val
301 305 310 315
Pro Leu Glu His Pro Thr Leu Glu Trp Phe Ala Ala Leu Gly Leu
316 320 325 330
Arg Trp Tyr Ala Leu Pro Ala Val Ser Asn Met Leu Leu Glu Ile
331 335 340 345
Gly Gly Leu Glu Phe Ser Ala Ala Pro Phe Ser Gly Trp Tyr Met
346 350 355 360
Ser Thr Glu Ile Gly Thr Arg Asn Leu Cys Asp Pro His Arg Tyr
361 365 370 375
Asn Ile Leu Glu Asp Val Ala Val Cys Met Asp Leu Asp Thr Arg
376 380 385 390
Thr Thr Ser Ser Leu Trp Lys Asp Lys Ala Ala Val Glu Ile Asn
391 395 400 405
Leu Ala Val Leu His Ser Phe Gln Leu Ala Lys Val Thr Ile Val
406 410 415 420
Asp His His Ala Ala Thr Val Ser Phe Met Lys His Leu Asp Asn
421 425 430 435
Glu Gln Lys Ala Arg Gly Gly Cys Pro Ala Asp Trp Ala Trp Ile
436 440 445 450
Val Pro Pro Ile Ser GLys er Leu Thr Pro Val Phe His Gln Glu
451 455 460 465
Met Val Asn Tyr Ile Leu Ser Pro Ala Phe Arg Tyr Gln Pro Asp
466 470 475 480
Pro Trp Lys GLy Ser Ala Thr Lys Gly Ala Gly Ile Thr Arg Lys
481 485 490 495
Lys Thr Phe Lys Glu Val Ala Asn Ala Val Lys Ile Ser Ala Ser
496 500 505 510
Leu Met Gly Thr Leu Met Ala Lys Arg Val Lys Ala Thr Ile Leu
511 515 510 525
Tyr Ala Ser Glu Thr Gly Arg Ala Gln Ser Tyr Ala Gln Gln Leu
526 530 535 540
Gly Arg Leu Phe Arg Lys Ala Phe Asp Pro Arg Val Leu Cys Met
541 545 550 555
Asp Glu Tyr Asp Val Val Ser Leu Glu His Glu Ala Leu Val Leu
556 560 565 570
Val Val Thr Ser Thr Phe Gly Asn Gly Asp Pro Pro Glu Asn Gly
571 575 580 585
Glu Ser Phe Ala Ala Ala Leu Met Glu Met Ser Gly Pro Tyr Asn
586 590 595 600
Ser Ser Pro Arg Pro Glu Gln His Lys Ser Tyr Lys Ile Arg Phe
601 605 610 615
Asn Ser Val Ser Cys Ser Asp Pro Leu Val Ser Ser Trp Arg Arg
616 620 625 630
Lys Arg Lys Glu Ser Ser Asn Thr Asp Ser Ala Gly Ala Leu Gly
631 635 640 645
Thr Leu Arg Phe Cys Val Phe Gly Leu GLy Ser Arg Ala Tyr Pro
646 650 655 660
His Phe Cys Ala Phe Ala Arg Ala Val Asp Thr Arg Leu Glu Glu
661 665 670 675
Leu Gly Gly Glu Arg Leu Leu Gln Leu Gly Gln Gly Asp Glu Leu
676 680 685 690
Cys Gly Gln Glu Glu Ala Phe Arg Gly Trp Ala Lys Ala Ala Phe
691 695 700 705
Gln Ala Ser Cys Glu Thr Phe Cys Val Gly Glu Glu Ala Lys Ala
706 710 715 720
Ala Ala Gln Asp Ile Phe Ser Pro Lys Arg Ser Trp Lys Arg Gln
721 725 730 735
Arg Tyr Arg Leu Ser Thr Gln Ala Glu Gly Leu Gln Leu Leu Pro
736 740 745 750
Gly Leu Ile His Val His Arg Arg Lys Met Phe Gln Ala Thr Val
751 755 760 765
Leu Ser Val Glu Asn Leu Gln Ser Ser Lys Ser Thr Arg Ala Thr
766 770 775 780
Ile Leu Val Arg Leu Asp Thr Ala Gly Gln Glu Gly Leu Gln Tyr
781 785 790 795
Gln Pro Gly Asp His Ile Gly Ile Cys Pro Pro Asn Arg Pro Gly
796 800 805 810
Leu Val Glu Ala Leu Leu Ser Arg Val Glu Asp Pro Pro Pro Pro
811 815 820 825
Thr Glu Ser Val Ala Val Glu Gln Leu Glu Lys GLys er Pro Gly
826 830 835 840
Gly Pro Pro Pro Ser Trp Val Arg Asp Pro Arg Leu Pro Pro Cys
841 845 850 855
Thr Leu Arg Gln Ala Leu Thr Phe Phe Leu Asp Ile Thr Ser Pro
856 860 865 870
Pro Ser Pro Arg Leu Leu Arg Leu Leu Ser Thr Leu Ala Glu Glu
871 875 880 885
Pro Ser Glu Gln Gln Glu Leu Glu Thr Leu Ser Gln Asp Pro Arg
886 890 895 900
Arg Tyr Glu Glu Trp Lys Trp Phe Arg Cys Pro Thr Leu Leu Glu
901 905 910 915
Val Leu Glu Gln Phe Pro Ser Val Ala Leu Pro Ala Pro Leu Leu
916 920 925 930
Leu Thr Gln Leu Pro Leu Leu Gln Pro Arg Tyr Tyr Ser Val Ser
931 935 940 945
Ser Ala Pro Asn Ala His Pro Gly Glu Val His Leu Thr Val Ala
946 950 955 960
Val Leu Ala Tyr Arg Thr Gln Asp Gly Leu Gly Pro Leu His Tyr
961 965 970 975
Gly Val Cys Ser Thr Trp Leu Ser Gln Leu Lys Thr Gly Asp Pro
976 980 985 990
Val Pro Cys Phe Ile Arg Gly Ala Pro Ser Phe Arg Leu Pro Pro
991 995 1000 1005
Asp Pro Tyr Val Pro Cys Ile Leu Val Gly Pro Gly Thr Gly Ile
1006 1010 1015 1020
Ala Pro Phe Arg Gly Phe Trp Gln Glu Arg Leu His Asp Ile Glu
1021 1025 1030 1035
Ser Lys Gly Leu Gln Pro Ala Pro Met Thr Leu Val Phe Gly Cys
1036 1140 1145 1050
Arg Cys Ser Gln Leu Asp His Leu Tyr Arg Asp Glu Val Gln Asp
1051 1155 1160 1065
Ala Gln Glu Arg Gly Val Phe Gly Arg Val Leu Thr Ala Phe Ser
1066 1170 1175 1080
Arg Glu Pro Asp Ser Pro Lys Thr Tyr Val Gln Asp Ile Leu Arg
1081 1185 1190 1095
Thr Glu Leu Ala Ala Glu Val His Arg Val Leu Cys Leu Glu Arg
1096 1100 1105 1110
Gly His Met Phe Val Cys Gly Asp Val Thr Met Ala Thr Ser Val
1111 1115 1120 1125
Leu Gln Thr Val Gln Arg Ile Leu Ala Thr Glu Gly Asp Met Glu
1126 1130 1135 1140
Leu Asp Glu Ala Gly Asp Val Ile Gly Val Leu Arg Asp Gln Gln
1141 1145 1150 1155
Arg Tyr His Glu Asp Ile Phe Gly Leu Thr Leu Arg Thr Gln Glu
1156 1160 1165 1170
Val Thr Ser Arg Ile Arg Thr Gln Ser Phe Ser Leu Gln Glu Arg
1171 1175 1180 1185
His Leu Arg Gly Ala Val Pro Trp Ala Phe Asp Pro Pro Gly Pro
1186 1190 1195 1200
Asp Thr Pro Gly Pro
1201 1205
```

Polyclonal antibodies to endothelial NO synthase may be obtained using the whole molecule of human endothelial NO synthase of the following sequence:

```
SEQ ID NO: 2
Met Gly Asn Leu Lys Ser Val Ala Gln Glu Pro Gly Pro Pro Cys
  1 5 10 15
Gly Leu Gly Leu Gly Leu Gly Leu Gly Leu Cys Gly Lys Gln Gly
  16 20 25 30
Pro Ala Thr Pro Ala Pro Glu Pro Ser Arg Ala Pro Ala Ser Leu
  31 35 40 45
Leu Pro Pro Ala Pro Glu His Ser Pro Pro Ser Ser Pro Leu Thr
  46 50 55 60
Gln Pro Pro Glu Gly Pro Lys Phe Pro Arg Val Lys Asn Trp Glu
  61 65 70 75
Val GLys er Ile Thr Tyr Asp Thr Leu Ser Ala Gln Ala Gln Gln
  76 80 85 90
Asp Gly Pro Cys Thr Pro Arg Arg Cys Leu GLys er Leu Val Phe
  91 95 100 105
Pro Arg Lys Leu Gln Gly Arg Pro Ser Pro Gly Pro Pro Ala Pro
106 110 115 120
Glu Gln Leu Leu Ser Gln Ala Arg Asp Phe Ile Asn Gln Tyr Tyr
121 125 130 135
Ser Ser Ile Lys Arg Ser GLys er Gln Ala His Glu Gln Arg Leu
136 140 145 150
Gln Glu Val Glu Ala Glu Val Ala Ala Thr Gly Thr Tyr Gln Leu
151 155 160 165
Arg Glu Ser Glu Leu Val Phe Gly Ala Lys Gln Ala Trp Arg Asn
166 170 175 180
Ala Pro Arg Cys Val Gly Arg Ile Gln Trp Gly Lys Leu Gln Val
181 185 190 195
Phe Asp Ala Arg Asp Cys Arg Ser Ala Gln Glu Met Phe Thr Tyr
196 200 205 210
Ile Cys Asn His Ile Lys Tyr Ala Thr Asn Arg Gly Asn Leu Arg
211 215 220 225
Ser Ala Ile Thr Val Phe Pro Gln Arg Cys Pro Gly Arg Gly Asp
226 230 235 240
Phe Arg Ile Trp Asn Ser Gln Leu Val Arg Tyr Ala Gly Tyr Arg
241 245 250 255
Gln Gln Asp GLy Ser Val Arg Gly Asp Pro Ala Asn Val Glu Ile
256 260 265 270
Thr Glu Leu Cys Ile Gln His Gly Trp Thr Pro Gly Asn Gly Arg
271 275 280 285
Phe Asp Val Leu Pro Leu Leu Leu Gln Ala Pro Asp Glu Pro Pro
286 290 295 300
Glu Leu Phe Leu Leu Pro Pro Glu Leu Val Leu Glu Val Pro Leu
301 305 310 315
Glu His Pro Thr Leu Glu Trp Phe Ala Ala Leu Gly Leu Arg Trp
316 320 325 330
Tyr Ala Leu Pro Ala Val Ser Asn Met Leu Leu Glu Ile Gly Gly
331 335 340 345
Leu Glu Phe Pro Ala Ala Pro Phe Ser Gly Trp Tyr Met Ser Thr
346 350 355 360
Glu Ile Gly Thr Arg Asn Leu Cys Asp Pro His Arg Tyr Asn Ile
361 365 370 375
Leu Glu Asp Val Ala Val Cys Met Asp Leu Asp Thr Arg Thr Thr
376 380 385 390
Ser Ser Leu Trp Lys Asp Lys Ala Ala Val Glu Ile Asn Val Ala
391 395 400 405
Val Leu His Ser Tyr Gln Leu Ala Lys Val Thr Ile Val Asp His
406 410 415 420
His Ala Ala Thr Ala Ser Phe Met Lys His Leu Glu Asn Glu Gln
421 425 430 435
Lys Ala Arg Gly Gly Cys Pro Ala Asp Trp Ala Trp Ile Val Pro
436 440 445 450
Pro Ile Ser GLys er Leu Thr Pro Val Phe His Gln Glu Met Val
451 455 460 465
Asn Tyr Phe Leu Ser Pro Ala Phe Arg Tyr Gln Pro Asp Pro Trp
466 470 475 480
Lys Gly Ser Ala Ala Lys Gly Thr Gly Ile Thr Arg Lys Lys Thr
481 485 490 495
Phe Lys Glu Val Ala Asn Ala Val Lys Ile Ser Ala Ser Leu Met
496 500 505 510
Gly Thr Val Met Ala Lys Arg Val Lys Ala Thr Ile Leu Tyr Gly
511 515 510 525
Ser Glu Thr Gly Arg Ala Gln Ser Tyr Ala Gln Gln Leu Gly Arg
526 530 535 540
Leu Phe Arg Lys Ala Phe Asp Pro Arg Val Leu Cys Met Asp Glu
541 545 550 555
Tyr Asp Val Val Ser Leu Glu His Glu Thr Leu Val Leu Val Val
556 560 565 570
Thr Ser Thr Phe Gly Asn Gly Asp Pro Pro Glu Asn Gly Glu Ser
571 575 580 585
Phe Ala Ala Ala Leu Met Glu Met Ser Gly Pro Tyr Asn Ser Ser
586 590 595 600
Pro Arg Pro Glu Gln His Lys Ser Tyr Lys Ile Arg Phe Asn Ser
601 605 610 615
Ile Ser Cys Ser Asp Pro Leu Val Ser Ser Trp Arg Arg Lys Arg
616 620 625 630
Lys Glu Ser Ser Asn Thr Asp Ser Ala Gly Ala Leu Gly Thr Leu
631 635 640 645
Arg Phe Cys Val Phe Gly Leu GLys er Arg Ala Tyr Pro His Phe
646 650 655 660
Cys Ala Phe Ala Arg Ala Val Asp Thr Arg Leu Glu Glu Leu Gly
661 665 670 675
Gly Glu Arg Leu Leu Gln Leu Gly Gln Gly Asp Glu Leu Cys Gly
676 680 685 690
Gln Glu Glu Ala Phe Arg Gly Trp Ala Gln Ala Ala Phe Gln Ala
691 695 700 705
Ala Cys Glu Thr Phe Cys Val Gly Glu Asp Ala Lys Ala Ala Ala
706 710 715 720
Arg Asp Ile Phe Ser Pro Lys Arg Ser Trp Lys Arg Gln Arg Tyr
721 725 730 735
Arg Leu Ser Ala Gln Ala Glu Gly Leu Gln Leu Leu Pro Gly Leu
736 740 745 750
Ile His Val His Arg Arg Lys Met Phe Gln Ala Thr Ile Arg Ser
751 755 760 765
Val Glu Asn Leu Gln Ser Ser Lys Ser Thr Arg Ala Thr Ile Leu
766 770 775 780
Val Arg Leu Asp Thr Gly Gly Gln Glu Gly Leu Gln Tyr Gln Pro
781 785 790 795
Gly Asp His Ile Gly Val Cys Pro Pro Asn Arg Pro Gly Leu Val
796 800 805 810
Glu Ala Leu Leu Ser Arg Val Glu Asp Pro Pro Ala Pro Thr Glu
811 815 820 825
Pro Val Ala Val Glu Gln Leu Glu Lys Gly Ser Pro Gly Gly Pro
826 830 835 840
Pro Pro Gly Trp Val Arg Asp Pro Arg Leu Pro Pro Cys Thr Leu
841 845 850 855
Arg Gln Ala Leu Thr Phe Phe Leu Asp Ile Thr Ser Pro Pro Ser
856 860 865 870
Pro Gln Leu Leu Arg Leu Leu Ser Thr Leu Ala Glu Glu Pro Arg
871 875 880 885
Glu Gln Gln Glu Leu Glu Ala Leu Ser Gln Asp Pro Arg Arg Tyr
886 890 895 900
Glu Glu Trp Lys Trp Phe Arg Cys Pro Thr Leu Leu Glu Val Leu
901 905 910 915
Glu Gln Phe Pro Ser Val Ala Leu Pro Ala Pro Leu Leu Leu Thr
916 920 925 930
Gln Leu Pro Leu Leu Gln Pro Arg Tyr Tyr Ser Val Ser Ser Ala
931 935 940 945
Pro Ser Thr His Pro Gly Glu Ile His Leu Thr Val Ala Val Leu
946 950 955 960
Ala Tyr Arg Thr Gln Asp Gly Leu Gly Pro Leu His Tyr Gly Val
961 965 970 975
Cys Ser Thr Trp Leu Ser Gln Leu Lys Pro Gly Asp Pro Val Pro
976 980 985 990
Cys Phe Ile Arg Gly Ala Pro Ser Phe Arg Leu Pro Pro Asp Pro
991 995 1000 1005
Ser Leu Pro Cys Ile Leu Val Gly Pro Gly Thr Gly Ile Ala Pro
1006 1010 1015 1020
Phe Arg Gly Phe Trp Gln Glu Arg Leu His Asp Ile Glu Ser Lys
1021 1025 1030 1035
Gly Leu Gln Pro Thr Pro Met Thr Leu Val Phe Gly Cys Arg Cys
1036 1140 1145 1050
Ser Gln Leu Asp His Leu Tyr Arg Asp Glu Val Gln Asn Ala Gln
1051 1155 1160 1065
Gln Arg Gly Val Phe Gly Arg Val Leu Thr Ala Phe Ser Arg Glu
1066 1170 1175 1080
Pro Asp Asn Pro Lys Thr Tyr Val Gln Asp Ile Leu Arg Thr Glu
1081 1185 1190 1095
Leu Ala Ala Glu Val His Arg Val Leu Cys Leu Glu Arg Gly His
1096 1100 1105 1110
Met Phe Val Cys Gly Asp Val Thr Met Ala Thr Asn Val Leu Gln
1111 1115 1120 1125
Thr Val Gln Arg Ile Leu Ala Thr Glu Gly Asp Met Glu Leu Asp
1126 1130 1135 1140
Glu Ala Gly Asp Val Ile Gly Val Leu Arg Asp Gln Gln Arg Tyr
1141 1145 1150 1155
His Glu Asp Ile Phe Gly Leu Thr Leu Arg Thr Gln Glu Val Thr
1156 1160 1165 1170
Ser Arg Ile Arg Thr Gln Ser Phe Ser Leu Gln Glu Arg Gln Leu
1171 1175 1180 1185
Arg Gly Ala Val Pro Trp Ala Phe Asp Pro Pro Gly Ser Asp Thr
1186 1190 1195 1200
Asn Ser Pro
1201 1203
```

To obtain polyclonal antibodies to endothelial NO synthase, it is also possible to use a fragment of endothelial NO synthase, selected, for example, from the following sequences:

```
SEQ ID NO: 3
Pro Trp Ala Phe
1192 1195
SEQ ID NO: 4
Gly Ala Val Pro
1189 1192
SEQ ID NO: 5
                                                                                                                Arg
                                                                                                              1185
His Leu Arg Gly Ala Val Pro Trp Ala Phe Asp Pro Pro Gly Pro
1186 1190 1195 1200
Asp Thr Pro Gly Pro
1201 1205
SEQ ID NO: 6
                                                                Ala Phe Asp Pro Pro Gly Pro
                                                                11941195 1200
Asp Thr Pro Gly Pro
1201 1205
SEQ ID NO: 7
His Leu Arg Gly Ala Val Pro Trp Ala Phe Asp
1186 1190 11951196
SEQ ID NO: 8
His Leu Arg Gly Ala Val Pro Trp Ala Phe Asp Pro Pro Gly Pro
1186 1190 1195 1200
Asp Thr Pro Gly Pro
1201 1205
```

The exemplary procedure for preparation of starting polyclonal antibodies to NO synthase may be described as follows: 7-9 days before blood sampling 1-3 intravenous injections are made to the rabbits to increase the level of polyclonal antibodies in the rabbit blood stream. Upon immunization, blood samples are taken to test the antibody level. Typically, the maximum level of the immune reaction of the soluble antigen is reached in 40-60 days after the first injection. After the termination of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube Product clots formed on the tube sides are removed with a wooden spatula, and a rod is placed into the clot in the tube center. The blood is then placed in a refrigerator for one night at the temperature of about 4° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. (or without addition NaN₃ —at temperature −70° C.). To separate the target antibodies to endothelial NO synthase from the antiserum, the following solid phase absorption sequence is suitable:

(a) 10 ml of antiserum of rabbit is diluted twofold with 0.15 M NaCl, after which 6.26 g Na₂SO₄, is added, mixed and incubated for about 12-16 hours at 4° C.;

(b) the sediment is removed by centrifugation, dissolved in 10 ml of phosphate buffer and dialyzed against the same buffer within one night at room temperature;

(c) after the sediment is removed by centrifugation, the solution is put on the column with DEAE-cellulose, counterbalanced by phosphate buffer;

(d) the antibody fraction is determined by measuring the optical density of eluate at 280 nanometers.

The isolated crude antibodies are purified using affine chromatography method by attaching the obtained antibodies to endothelial NO synthase located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to endothelial NO synthase is 0.5 to 5.0 mg/ml, preferably, 2.0 to 3.0 mg/ml.

The polyclonal antibodies to prostate specific antigen may also be obtained by a similar methodology to the methodology described for endothelial NO synthase antibodies using an adjuvant. The entire molecule of human prostate specific antigen of the following sequence may be used as immunogen (antigen) for rabbits' immunization:

```
SEQ ID NO: 9
Met Trp Val Pro Val Val Phe Leu Thr Leu Ser Val Thr Trp Ile
  1 5 10 15
Gly Ala Ala Pro Leu Ile Leu Ser Arg Ile Val Gly Gly Trp Glu
  16 20 25 30
Cys Glu Lys His Ser Gln Pro Trp Gln Val Leu Val Ala Ser Arg
  31 35 40 45
Gly Arg Ala Val Cys Gly Gly Val Leu Val His Pro Gln Trp Val
  46 50 55 60
Leu Thr Ala Ala His Cys Ile Arg Asn Lys Ser Val Ile Leu Leu
  61 65 70 75
Gly Arg His Ser Leu Phe His Pro Glu Asp Thr Gly Gln Val Phe
  76 80 85 90
Gln Val Ser His Ser Phe Pro His Pro Leu Tyr Asp Met Ser Leu
  91 95 100 105
Leu Lys Asn Arg Phe Leu Arg Pro Gly Asp Asp Ser Ser His Asp
106 110 115 120
Leu Met Leu Leu Arg Leu Ser Glu Pro Ala Glu Leu Thr Asp Ala
121 125 130 135
Val Lys Val Met Asp Leu Pro Thr Gln Glu Pro Ala Leu Gly Thr
136 140 145 150
Thr Cys Tyr Ala Ser Gly Trp Gly Ser Ile Glu Pro Glu Glu Phe
151 155 160 165
Leu Thr Pro Lys Lys Leu Gln Cys Val Asp Leu His Val Ile Ser
166 170 175 180
Asn Asp Val Cys Ala Gln Val His Pro Gln Lys Val Thr Lys Phe
181 185 190 195
Met Leu Cys Ala Gly Arg Trp Thr Gly Gly Lys Ser Thr Cys Ser
196 200 205 210
Gly Asp Ser Gly Gly Pro Leu Val Cys Asn Gly Val Leu Gln Gly
211 215 220 225
Ile Thr Ser Trp Gly Ser Glu Pro Cys Ala Leu Pro Glu Arg Pro
226 230 235 240
Ser Leu Tyr Thr Lys Val Val His Tyr Arg Lys Trp Ile Lys Asp
241 245 250 255
Thr Ile Val Ala Asn Pro
256 260 261
```

To obtain polyclonal antibodies to prostate specific antigen, it is also possible to use a fragment of prostate specific antigen, selected, for example, from the following sequences:

```
SEQ ID NO: 10
                                                                                                Thr Lys Phe
                                                                                                193 195
Met Leu Cys Ala Gly Arg Trp Thr Gly Gly Lys Ser Thr
196 200 205 208
SEQ ID NO: 11
Gly Asp Ser Gly Gly Pro Leu Val Cys Asn Gly Val Leu Gln Gly
211 215 220 225
Ile Thr Ser Trp Gly Ser Glu Pro Cys Ala Leu Pro Glu Arg Pro
226 230 235 240
Ser
241
SEQ ID NO: 12
                                                                Pro Gln Lys Val Thr Lys Phe
                                                                189 190 195
Met Leu Cys Ala Gly
196 200
SEQ ID NO: 13
                                                Ile Arg Asn Lys Ser Val Ile Leu Leu
                                                  67 70 75
Gly Arg His Ser Leu Phe His Pro Glu Asp Thr Gly Gln Val Phe
  76 80 85 90
Gln Val Ser His Ser Phe Pro His Pro Leu Tyr Asp Met Ser Leu
  91 95 100 105
Leu Lys Asn Arg Phe Leu Arg Pro Gly Asp Asp Ser Ser His Asp
106 110 115 120
Leu Met Leu Leu Arg Leu Ser Glu Pro Ala Glu Leu Thr Asp Ala
121 125 130 135
Val Lys Val Met Asp Leu Pro Thr Gln Glu Pro Ala Leu Gly Thr
136 140 145 150
Thr Cys Tyr Ala Ser Gly Trp Gly Ser Ile Glu Pro Glu Glu Phe
151 155 160 165
Leu Thr Pro Lys Lys Leu Gln Cys Val Asp Leu His Val Ile Ser
166 170 175 180
Asn Asp Val Cys Ala Gln Val His Pro Gln
181 185 190
SEQ ID NO: 14
        Arg His Ser Leu Phe His Pro Glu Asp Thr Gly Gln Val Phe
          77 80 85 90
Gln Val Ser His Ser Phe Pro His Pro
  91 95 99
SEQ ID NO: 15
                                                                                Tyr Asp Met Ser Leu
                                                                                101 105
Leu Lys Asn Arg Phe Leu Arg Pro Gly Asp Asp Ser Ser His Asp
106 110 115 120
Leu Met Leu Leu Arg
121 125
SEQ ID NO: 16
        Met Leu Leu Arg Leu Ser Glu Pro Ala Glu Leu Thr Asp Ala
        122 125 130 135
SEQ ID NO: 17
                                Val Val Phe Leu Thr Leu Ser Val Thr Trp Ile
                                  5 10 15
Gly Ala Ala Pro Leu Ile Leu Ser Arg Ile
  16 20 25
SEQ ID NO: 18
        Lys Asn Arg Phe Leu Arg Pro Gly Asp Asp Ser Ser His Asp
        107 110 115 120
Leu Met Leu Leu Arg Leu Ser Glu Pro Ala Glu Leu Thr Asp Ala
121 125 130 135
Val Lys Val Met Asp Leu Pro Thr Gln Glu Pro Ala Leu Gly Thr
136 140 145 150
Thr Cys Tyr Ala Ser Gly Trp Gly Ser Ile Glu Pro Glu Glu Phe
151 155 160 165
Leu Thr Pro Lys Lys Leu Gln Cys Val Asp Leu His Val Ile Ser
166 170 175 180
Asn Asp Val Cys Ala Gln Val His Pro Gln Lys Val Thr Lys Phe
181 185 190 195
Met Leu Cys Ala Gly
196 200
SEQ ID NO: 19
                                                                        Ile Val Gly Gly Trp Glu
                                                                          25 30
Cys Glu Lys His Ser Gln Pro Trp Gln Val Leu Val Ala Ser Arg
  31 35 40 45
Gly Arg Ala Val Cys Gly Gly Val Leu Val His Pro Gln Trp Val
  46 50 55 60
Leu Thr Ala Ala His Cys Ile Arg Asn Lys Ser Val Ile Leu Leu
  61 65 70 75
Gly Arg His Ser Leu Phe His Pro Glu Asp Thr Gly Gln Val Phe
  76 80 85 90
Gln Val Ser His Ser Phe Pro His Pro Leu Tyr Asp Met Ser Leu
  91 95 100 105
Leu Lys Asn Arg Phe Leu Arg Pro Gly Asp Asp Ser Ser His Asp
106 110 115 120
Leu Met Leu Leu Arg Leu Ser Glu Pro Ala Glu Leu Thr Asp Ala
121 125 130 135
Val Lys Val Met Asp Leu Pro Thr Gln Glu Pro Ala Leu Gly Thr
136 140 145 150
Thr Cys Tyr Ala Ser Gly Trp Gly Ser Ile Glu Pro Glu Glu Phe
151 155 160 165
Leu Thr Pro Lys Lys Leu Gln Cys Val Asp Leu His Val Ile Ser
166 170 175 180
Asn Asp Val Cys Ala Gln Val His Pro Gln Lys Val Thr Lys Phe
181 185 190 195
Met Leu Cys Ala Gly Arg Trp Thr Gly Gly Lys Ser Thr Cys Ser
196 200 205 210
Gly Asp Ser Gly Gly Pro Leu Val Cys Asn Gly Val Leu Gln Gly
211 215 220 225
Ile Thr Ser Trp Gly Ser Glu Pro Cys Ala Leu Pro Glu Arg Pro
226 230 235 240
Ser Leu Tyr Thr Lys Val Val His Tyr Arg Lys Trp Ile Lys Asp
241 245 250 255
Thr Ile Val Ala Asn Pro
256 260 261
```

The activated-potentiated form of each component of the combination may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to prostate specific antigen with the concentration of 3.0 mg/ml is diluted in 99 parts of neutral aqueous or aqueous-alcohol solvent (preferably, 15%-ethyl alcohol) and then vertically shaked many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies with the concentration of 3.0 mg/ml in 99 parts of a neutral solvent in different containers, which is equivalent to the centesimal homeopathic dilution C12. Similar procedures with the relevant dilution factor are performed to obtain the desired dilutions. The intermediate dilutions may be tested in a desired biological model to check activity. The preferred activated potentiated form for antibodies comprising the combination of the invention is a C12, C30 and C200 dilutions for each activated-potentiated form. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D20, C30, C100 or C12, C30, C50 or C12, C30, C200, etc.), the efficiency of which is determined experimentally by testing the dilution in a suitable biological model, for example, in models described in the examples herein.

In the course of potentiation and concentration decrease, the vertical shaking may be substituted for external exposure to ultrasound, electromagnetic field or any similar external impact procedure accepted in the homeopathic art.

The solid unit dosage form of the pharmaceutical composition of the invention may be prepared by using impregnating a solid, pharmaceutically acceptable carrier with the mixture of the activated potentiated form aqueous or aqueous-alcohol solutions of active components that are mixed, primarily in 1:1:1 ratio and used in liquid dosage form. Alternatively, the carrier may be impregnated consecutively with each requisite dilution.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose and magnesium stearate.

The example of preparation of the solid unit dosage form is set forth below. To prepare the solid oral form, 100-300 μm granules of lactose are impregnated with aqueous or aqueous-alcoholic solutions of the activated potentiated form of antibodies to prostate specific antigen and activated-potentiated form of antibodies to endothelial NO synthase in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Hüttlin Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch—XL 400 tablet press) to form 150 to 500 mg round pills, preferably, 300 mg. After tableting, 300 mg pills are obtained that are saturated with aqueous-alcohol solution (3.0-6.0 mg/pill) of the combination of the activated-potentiated form of antibodies. Each component of the combination used to impregnate the carrier is in the form of a mixture of centesimal homeopathic dilutions, preferably, C12, C30 and C200.

While the invention is not limited to any specific theory, it is believed that the activated-potentiated form of the antibodies described herein do not contain the molecular form of the antibody in an amount sufficient to have biological activity attributed to such molecular form. The biological activity of the combination drug (combination pharmaceutical composition) of the invention is amply demonstrated in the appended examples.

The invention is further illustrated with reference to the appended non-limiting examples.

### EXAMPLES

### Example 1

The experimental study looked at the efficacy of activated-potentiated rabbit polyclonal affinity purified on antigen antibodies to prostate specific antigen (anti-PSA) and to endothelial NO synthase (anti-eNOS) in ultra-low doses (ULD), obtained by a ultra dilution of the initial matrix solution (with 2.5 mg/ml concentration) 100¹², 100³⁰, 100²⁰⁰ times, equivalent to a mixture of centesimal homeopathic dilutions C12, C30, C200 (ULD anti-PSA+anti-eNOS) in a model of a benign prostatic hyperplasia (BPH) in rats.

BPH is one of the widely spread urologic disorders in men. The risk of development of this disorder increases with age: approximately 10% of men over 40 years old have BPH; after 60 years old their number increases up to 30-40%. Benign Prostatic Hypoplasia can be defined as hyperplasia of prostate tissues, accompanied by urination problems (including increased urination frequency, false urges, nocturia, weak or intermittent urine stream, and a sensation of incomplete bladder emptying). BPH symptoms significantly affect quality of life in patients. This is a progressing disease, and without an adequate treatment can lead to such serious complications as acute urinary retention, disruption of the voiding cycle, kidney failure.

One of BPH models in rodents is hormone-induced prostate inflammation, causing its enlargement. To achieve that, hyperprolactinemia in induced in rodents, by injecting them with sulpiride in the amount of 40 mg/kg during 60 days intraperitoneally (Coppenolle V. F. et al. Effects of hyperprolactinemia on rat prostate growth: evidence of androgeno-dependence//Am. J. Physiol. Endocrinol. Metab. 2001. 280: E 120-E129).

During the study on effectiveness of ultra low doses of anti-PSA+anti-eNOS in this model of benign prostate hyperplasia (BPH) in rats, 30 male wistar rats were used (8 months, weighing 600-650 g). Ten rats were intact. The rest were induced with prostatic hyperplasia (sulpiride was injected intraperitoneally in the amount of 40 mg/kg for 60 days) and simultaneously with sulpiride distilled water was added endogasdtrically (control group, n=10; 10 ml/kg) or ultra low doses of anti-PSA+anti-eNOS (n=10; 10 ml/kg).

In 60 days, the prostatic weight coefficient was measured (ratio between the weight of prostate and the weight of the rodent), prostate volume and density, stromal-epithelial ratio in the prostate (value representing ratio between connective and secreting tissues in the organ), as well as a concentration in the blood of prolactin receptor (indirect indicator of hyperprolactinemia).

As a result of injecting sulpiride, the rats developed hyperprolactinemia (the level of prolactine receptor, controlling prolactine and growth hormone, increased in the control group by 83.3% compared to the intact group), causing an increase of weight coefficient of prostate by 51.9% (p<0.05) and its volume by 33.3% (p<0.05), compared to the control group (Table 1). At the same time, replacement of secreting tissue with connective takes place (stromal-epithelial ratio would decrease by 29.6%, p<0.05), indicating an inflammation.

Introduction of ultra low doses of anti-PSA+anti-eNOS in rats with prostatic hyperplasia led to reducing levels of prolactin receptor (by 40.6%, p<0.05, compared to the control group), decreasing the prostate weight coefficient and volume (by 22.0% and 41.7%, correspondingly, p<0.05), and also disappearance of inflammation (stromal-epithelial ratio did not differ from intact rats).

TABLE 1
Effect of ultra low doses of anti-PSA + anti-eNOS on the prostate under
the conditions of hormone induced prostatic hyperplasia.

| | | | | Content of |
|---|---|---|---|---|
| | Weight | | | receptor |
| | coefficient of | Volume of | Stromal- | toward |
| | prostate, | prostate, | epithelial | prolactin, |
| | mg/g | cm³ | ratio | ng/ml |
| Intact | 0.27 ± 0.01 | 0.09 ± 0.01 | 1.25 ± 0.09 | 0.090 ± 0.010 |
| Control | 0.41 ± 0.02* | 0.12 ± 0.01* | 0.88 ± 0.07* | 0.165 ± 0.033 |
| ULD anti- | 0.32 ± 0.02*^{#} | 0.07 ± 0.01^{#} | 1.19 ± 0.05^{#} | 0.098 ± 0.005 |
| PSA + | | | | |
| anti-eNOS | | | | |

Note:
*difference vs intact is significant at p < 0.05;
^{#}difference vs control is significant at p < 0.05

Therefore, the proposed pharmaceutical product of ultra low doses of anti-PSA+anti-eNOS, is effective under the conditions of an experimental model of benign prostatic hyperplasia (hormone-induced inflammation).

### Example 2

To study properties of the proposed pharmaceutical composition in the treatment of patients with a benign prostatic hyperplasia, 300 mg pills were used, saturated with the pharmaceutical composition containing water-alcohol solutions (6 mg/pill) of activated-potentiated rabbit polyclonal affinity purified antibodies to prostate specific antigen (anti-PSA) and endothelial NO synthase (anti-eNOS) in ultra low doses (ULD), produced by ultra dilution of the initial matrix solution 100¹², 100³⁰, 100²⁰⁰ times, equivalent to the mixture of centesimal homeopathic dilutions C12, C30, C200 (ULD anti-PSA+anti-eNOS), and 300 mg pills, saturated with the pharmaceutical composition containing water-alcohol solutions (3 mg/pill) of activated potentiated rabbit polyclonal affinity purified antibodies to prostate specific antigen in ultra low doses (ULD), obtained by an ultra dilution of the initial matrix solution 100¹², 100³⁰, 100²⁰⁰ times, equivalent to the mixture of centesimal homeopathic dilutions C12, C30, C200 (ULD anti-PSA).

Benign prostatic hyperplasia (BPH) is one of the most frequently occurring disorders in males (Bruskewitz R. C., 2003; Rosen R., 2003): on the one hand, epidemiological studies, carried out in Russia, point to a gradual increase in frequency of BPH from 11.3% in 40-49 year olds to 81.4% in 80 year olds (Gorilovskiy, L. M., 1999); on the other hand, demographic studies conducted by WHO confirm a significant increase in the population over 60 years old, surpassing any other age group growth.

The main symptoms of benign prostatic hyperplasia are lower urinary tract symptoms, which can cause significant discomfort and decrease quality of life (Bruskewitz R. C., 2003; Lepor H., 2004; O'Leary M. P., 2005). In severe cases, the disease can be accompanied by complications, such as acute urinary retention, urinary tract infection, erythruria, kidney failure (Stepanov, V. N., 1999; Jacobsen S. J., 1997; Lepor H., 2004). BPH is also associated with development of erectile dysfunction in patients (Bruskewitz R. C., 2003; Daly M P, 2005).

An open-label comparative parallel group study of efficacy and safety the of pharmaceutical compositions containing ULD anti-PSA+ULD anti-eNOS and ULD anti-PSA in ameliorating urinary disturbances to caused by benign prostatic hyperplasia (BPH), included 40 patients selected in accordance with inclusions/exclusions criteria. Patients were randomized in 2 groups, one group received 1 pill 3 times per day during 12 weeks (n=21) of a ULD anti-PSA+anti-eNOS, and another one 1 pill 3 times per day during 12 weeks (n=19) of a ULD anti-PSA. The groups were comparable in age, severity of BPH symptoms, urination parameters and prostate volume.

The study included patients over 45 years old with a history of BPH with corresponding symptoms of lower urinary tract for no less than 6 months, IPSS≧13, prostate volume according to transrectal ultrasonography ≧30 cm3, with maximum urinary flow speed of ≧4 ml/s and ≦15 ml/sc and minimum residual urine volume equal to 125 ml, with PSA level ≦4 ng/ml. A necessary inclusion criterion was absence of intake of the following medications in the medical records: finasteride, dutasteride, or other experimental drug 6 months prior to inclusion in the study, α1-adrenoreceptor blockers and herbal medications 4 weeks prior to the inclusion into the study, any inhibitors of phosphodiesterase type 5 and other erectile dysfunction treatments 4 weeks prior to the inclusion into the study.

The study did not include patients undergone invasive methods of treatment of BPH, including transurethral prostatic resection, thermotherapy, transurethral needle ablation, stent angioplasty and other; with malignant oncological disease, acute urination delay, bladder stones, urethral stricture, Marion's disease, genitourinary system infections in the phase of active inflammation and others.

Clinical efficacy of pharmaceutical compositions was assessed by the improvement of clinical symptoms of lower urinary tract, evaluated using IPSS questionnaire (International Prostate Symptom Score), urination parameters (maximum and average urinary flow speed, urination volume, volume of residual urine) and prostate volume based on the data of transurethral ultrasound (TU), and also erectile function was evaluated based on the data obtained from IIEF questionnaire (International Index of Erectile Function). Results of the study are shown in tables 2 and 3.

TABLE 2

| | ULD anti-PSA | | | | ULD anti-PSA + ULD anti-eNOS | | | |
|---|---|---|---|---|---|---|---|---|
| | | | 12 | | | | 12 | |
| | n/N | In., | weeks., | Δ, | n/N | In., | weeks., | Δ, |
| | (%)¹ | aver. | aver. | aver. | (%)¹ | aver. | aver. | aver. |
| IPSS, score | 19/19 | 17.8 | 11.9 | −5.9 | 20/21 | 16.0 | 10.5 | −5.6 |
| | (100.0) | | | | (95.2) | | | |
| QoL/, score | 19/19 | 3.4 | 2.4 | −1.0 | 20/21 | 3.4 | 2.3 | −1.1 |
| (quality of | (100.0) | | | | (95.2) | | | |
| life) | | | | | | | | |
| IIEF, score | 2/19 | 17.8 | 18.6 | 0.8 | 4/21 | 17.5 | 18.9 | 1.4 |
| | (10.5) | | | | (19.0) | | | |
| Qmax, ml/s | 16/19 | 10.8 | 13.1 | 2.2 | 15/21 | 11.7 | 13.7 | 2.0 |
| (maximum | (84.2) | | | | (71.4) | | | |
| urine rate) | | | | | | | | |
| Qave, ml/s | 15/19 | 5.8 | 7.1 | 1.3 | 18/21 | 5.8 | 7.1 | 1.3 |
| (average | (78.9) | | | | (85.7) | | | |
| urine rate) | | | | | | | | |
| V, ml | 10/19 | 218.6 | 206.8 | −11.8 | 15/21 | 203.7 | 252.0 | 48.3 |
| (volume of | (52.6) | | | | (71.4) | | | |
| urination) | | | | | | | | |
| RV, ml | 15-19 | 23.6 | 19.4 | −4.3 | 14/21 | 19.1 | 14.1 | −5.0 |
| (residual | (78.9) | | | | (66.6) | | | |
| volume of | | | | | | | | |
| urine) | | | | | | | | |
| PV, cm³ | 18/19 | 55.9 | 48.9 | −7.0 | 15/21 | 57.0 | 52.4 | −4.6 |
| (prostate | (94.7) | | | | (71.4) | | | |
| volume) | | | | | | | | |

¹the numerator is a number of patients (n) showing improvement, denominator is total number of patients in the study (N).

TABLE 3
Dynamics of subscales of obstructive and irritative symptoms, and
question 7 of IPSS questionnaire

| | ULD anti-PSA | | ULD anti-PSA + anti-eNOS | |
|---|---|---|---|---|
| | M ± SD Visit 1 | M ± SD Visit 2 | M ± SD Visit 1 | M ± SD Visit 2 |
| Obstructive | 10.0 ± 3.02# | 6.5 ± 2.81*** | 8.2 ± 2.96 | 6.0 ± 3.39** |
| Irrit. | 7.5 ± 2.21& | 5.3 ± 1.90*** | 7.8 ± 2.16& | 4.5 ± 2.34*** |
| 7^{th} question | 2.1 ± 0.78 | 1.9 ± 0.75 | 2.3 ± 0.90 | 1.4 ± 0.98*** |
| Obstr., %² | | −33.4 ± 26.85 | | −25.2 ± 34.50 |
| Irrit., %² | | −28.2 ± 1730 | | −40.3 ± 30.35 |
| 7^{th} question, | | −2.0 ± 49.61## | | −37.7 ± 39.23 |
| %² | | | | |

*p < 0.05 vs baseline;
**p < 0.01 vs vaseline;
***p < 0.001 vs baseline
##p < 0.01 vs ULD anti-PSA
²shows decrease compared to the baseline in %, average group value

The given data confirm that both ULD anti-PSA, and ULD anti-PSA+ULD anti-eNOS were used to effectively treat symptoms of lower urinary tract, increase average and maximum urinary flow speed, improve quality of life of patients (Table 2). The course was not long (12 weeks), therefore, a decrease in prostate volume was not observed in any study group. ULD anti-PSA did not affect the volume of urination, which increased only in 52.6% patients, on average the group showed some statistically insignificant decrease of urination volume by 11.8 ml (5.4%) compared to the baseline values. At the same time, patients, treated with ULD anti-PSA+ULD anti-eNOS, showed an increase in urination volume in 71.4%, and on average, an increase in volume was 48.3 ml (23.7%) compared to the baseline.

An analysis of dynamics of obstructive and irritative symptoms according to IPSS subscales as well as nucturia evidence (question 7 of IPSS) showed that both pharmaceutical compositions contributed to a decrease of obstruction and irritative symptoms, and also a decrease of nucturia symptoms. At the same time, a ULD anti-PSA+anti-eNOS was more effective compared to a ULD anti-PSA in decreasing irritative symptoms of lower urinary tract (28.2% vs. 40.3%, p<0.05) and nighttime urination urges (2.0% vs. 37.7%).

It should be noted, that ULD anti-PSA+ULD anti-eNOS is also more effective compared to ULD anti-PSA in improving erectile function in patients. In ULD anti-PSA+ULD anti-eNOS group, the total IIEF (International Index of Erectile Dysfunction) score increased by 19% in patients (in ULD anti-PSA group by 10.5%), an average increase of IIEF score in ULD anti-PSA+ULD anti-eNOS group was 8% vs 4.5% in a ULD anti-PSA group.

The pharmaceutical compositions showed excellent safety profile, no adverse effects related to the administered medications were observed in the course of study.

Therefore, ULD anti-PSA+ULD anti-eNOS showed better efficacy compared to that of ULD anti-PSA in treating urination problems caused by benign prostatic hyperplasia. In addition, a greater positive effect of ULD anti-PSA+ULD anti-eNOS on erectile function of patients compared to ULD anti-PSA was revealed.

### Example 3

The experimental study looked at the efficacy of activated-potentiated rabbit polyclonal affinity purified on antigen antibodies to prostate specific antigen (anti-PSA) and to endothelial NO synthase (anti-eNOS) in ultra-low doses (ULD), obtained by a ultra dilution of the initial matrix solution (with 2.5 mg/ml concentration) 100¹², 100³⁰, 100²⁰⁰ times, equivalent to a mixture of centesimal homeopathic dilutions C12, C30, C200 (ULD anti-PSA+anti-eNOS) in a model of chronic prostatitis in rats.

### Efficacy in a Model of Prostatitis in Rats

Inflammatory diseases of prostate are among the most important urinary tract diseases [Mazo E B, Dmitriev D G, 2001; Scheplev P A et al., 2007]. The most common of them is prostatitis. Non-bacterial forms of prostatitis occur 8 times more frequently than bacterial ones [VA Smirnov, 2006]. The incidence of chronic prostatitis, non-urethral infection and other urological diseases in 40-50 year old men is 30-40%. This disease is rather difficult to treat, because even when subjective symptoms are disappeared and laboratory signs inflammation are reduced the morphological changes in the glandular tissue and prostate stroma are still present [VA Smirnov, 2006].

An animal models of non-bacterial prostatitis is partial prostate obstruction by applying silk ligature was used.

Sixty male rats of Wistar (2 months old, 250 g weight) were included in the study. Twelve rats were intact. To induce prostatitis the prostate of other rats were sewed with silk thread under general anesthesia (thiopental 60 mg/kg intraperitoneally). The rats were administered with ULD anti-PSA+anti-eNOS (5 ml/kg, 7.5 ml/kg or 10 ml/kg) or distilled water (control, 10 ml/kg) for 1.5 months starting 1 month after the surgery. After 2.5 months of prostatitis induction prostate was weighed, prostate coefficient was calculated; and prostate volume and density were assessed. A histological study of prostate from 6 animals of each group were performed: area of collagen fibers in the connective tissue (Sc, index sclerotic processes in the gland), the area of prostate epithelial acini (Se, index of atrophic processes in the gland), the area of the lumen of acini (SI, index of secretory activity of the gland).

After applying silk ligature to the rat's prostate an inflammation in the prostate was developed, which led to a significant increase in prostate weight coefficient in 25%, and the prostate density in 14% as well as to sclerotic changes in the prostate tissue (Sc increased 3.1-fold) compared with intact animals (Table 4.) The administration of ULD anti-PSA+anti-eNOS in all doses did not lead to significant reduction of prostate weight coefficient and prostate density, but significantly (almost down to that of intact animals) reduced the area of collagen fibers, which indicated a decrease in the inflammatory process in the gland (Table 4). Besides, ULD anti-PSA+anti-eNOS in a dose of 10 ml/kg increased the area of the lumen of acini, thus indicating increased secretory activity of the prostate (in 19.5%, p=0.055, Table 1).

Thus, ULD anti-PSA+anti-eNOS exerted anti-inflammatory activity in a model of non-bacterial prostatitis in rats.

TABLE 4

| | | | | ULD anti- | |
|---|---|---|---|---|---|
| | | | ULD anti- | PSA + anti- | ULD anti-PSA + |
| | | | PSA + anti- | eNOS | anti-eNOS |
| | Intact | Control | eNOS, 5 ml/kg | 7.5 ml/kg | 10 ml/kg |
| Weight | 0.80 ± 0.02 | 1.00 ± 0.07* | 1.03 ± 0.06* | 1.10 ± 0.04* | 0.99 ± 0.06* |
| coefficient | | | | | |
| of | | | | | |
| prostate. | | | | | |
| mg/g | | | | | |
| Prostate | 0.38 ± 0.02 | 0.39 ± 0.03 | 0.43 ± 0.02 | 0.44 ± 0.03 | 0.40 ± 0.03 |
| volume | | | | | |
| Prostate | 0.93 ± 0.04 | 1.06 ± 0.03* | 1.05 ± 0.03* | 1.05 ± 0.01* | 1.01 ± 0.03* |
| density | | | | | |
| Sc | 1.45 ± 0.38 | 4.51 ± 0.38* | 2.12 ± 0.27### | 2.12 ± 0.30### | 2.50 ± 0.41*### |
| Se | 18.45 ± 1.15 | 15.85 ± 1.52 | 14.49 ± 1.53* | 17.87 ± 2.23 | 12.20 ± 0.62* |
| Sl | 50.17 ± 3.61 | 51.73 ± 3.76 | 51.23 ± 2.63 | 59.30 ± 2.28* | 61.82 ± 2.62*# |

Note:
*difference is significant vs intact at p < 0.05;
#, ###difference is significant vs control at p < 0.05; —p < 0.001 respectively

### REFERENCES

1. Mazo E B, Dmitriev D G The clinical effect of the drug “Prostamol Uno” in patients with benign prostatic hyperplasia and chronic prostatitis//Urology.—2001.—N^{o} 5.—P. 38-41.
2. Smirnov V A Drug therapy is the chronic prostatitis//Farmindeks-Praktik.—2006.—Issue 10.—P. 46-55.
3. Scheplev P A Strachinsky L S, Rafalsky V. Prostatitis//M.: Medpress-inform, 2007.—224 pp.

What is claimed is:

1. A pharmaceutical composition comprising a) an activated-potentiated form of an antibody to prostate-specific antigen, and b) an activated-potentiated form of an antibody to endothelial NO-synthase.

2. The combination pharmaceutical composition of claim 1, which further comprises a solid carrier, wherein said activated-potentiated form an antibody to prostate-specific antigen and said activated-potentiated form of an antibody to endothelial NO-synthase are impregnated onto said carrier.

3. The combination pharmaceutical composition of claim 2, which is in the form of a tablet.

4. The combination pharmaceutical composition of claim 1, wherein said activated-potentiated form of an antibody to prostate-specific antigen is in the form of a mixture of C12, C30, and C200 homeopathic dilutions.

5. The combination pharmaceutical composition of claim 4, wherein said mixture of C12, C30, and C200 homeopathic dilutions is impregnated onto a solid carrier.

6. The combination pharmaceutical composition of claim 1, wherein said activated-potentiated form of an antibody to endothelial NO-synthase is in the form of a mixture of C12, C30, and C200 homeopathic dilutions.

7. The combination pharmaceutical composition of claim 6, wherein said mixture of C12, C30, and C200 homeopathic dilutions is impregnated onto a solid carrier.

8. The combination pharmaceutical composition of claim 1, wherein the activated-potentiated form of an antibody to prostate-specific antigen is a monoclonal, polyclonal or natural antibody.

9. The combination pharmaceutical composition of claim 8, wherein the activated-potentiated form of an antibody to prostate-specific antigen is a polyclonal antibody.

10. The combination pharmaceutical composition of claim 1, wherein the activated-potentiated form of an antibody to endothelial NO-synthase is a monoclonal, polyclonal or natural antibody.

11. The combination pharmaceutical composition of claim 10, wherein the activated-potentiated form of an antibody to endothelial NO-synthase is a polyclonal antibody.

12. The combination pharmaceutical composition of claim 1, wherein the activated-potentiated form of an antibody to endothelial endothelial NO-synthase is to the entire molecule of endothelial NO-synthase having the sequence SEQ ID NO: 1 or SEQ ID NO: 2.

13. The combination pharmaceutical composition of claim 1, wherein the activated-potentiated form of an antibody to endothelial NO-synthase is to a fragment of NO-synthase having a sequence selected from group consisting of SEQ ID NO. 3, SEQ ID NO: 4, SEQ ID NO: 5, SEQ ID NO: 6, SEQ ID NO: 7, and SEQ ID NO: 8.

14. The combination pharmaceutical composition of claim 1, wherein the activated-potentiated form of an antibody to prostate-specific antigen having sequence selected from the group consisting of, SEQ ID NO: 10, SEQ ID NO: 11, SEQ ID NO. 11, SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

15. The combination pharmaceutical composition of claim 1, wherein the activated-potentiated form of an antibody to prostate-specific antigen is to the entire molecule having SEQ ID NO: 9.

16. The combination pharmaceutical composition of claim 1, wherein said activated-potentiated forms of an antibody are prepared by successive centesimal dilutions coupled with shaking of every dilution.

17. A method of treating benign prostatic hyperplasia comprising administering the pharmaceutical composition of claim 1 to the patent in need thereof.

18. A method of treating erectile dysfunction comprising administering the pharmaceutical composition of claim 1 to the patent in need thereof.

19. A method of treating combined occurrence of benign prostatic hyperplasia and erectile dysfunction comprising administering the pharmaceutical composition of claim 1 to the patent in need thereof.

20. A method of treating chronic prostatitis comprising administering the pharmaceutical composition of claim 1 to the patent in need thereof.
